(19) United States
(12) Patent Application Publication
(10) Pub. No.: US 2024/0410002 A1
(43) Pub. Date: Dec. 12, 2024

(54) POLYCYSTIC KIDNEY DISEASE DIAGNOSIS AND TREATMENT BASED ON DETECTION OF PKD1/PKD2 VARIANTS

(71) Applicant: Helix, Inc., San Mateo, CA (US)

(72) Inventors: Natalie Telis, San Carlos, CA (US); Elizabeth Cirulli Rogers, Lakeside, CA (US); Cassie Hajek, Sioux Falls, SD (US)

(21) Appl. No.: 18/470,759

(22) Filed: Sep. 20, 2023

Related U.S. Application Data
(60) Provisional application No. 63/471,589, filed on Jun. 7, 2023.

(51) Int. Cl. C12Q 1/6883; C12Q 1/6869

(52) U.S. Cl. CPC C12Q 1/6883; C12Q 1/6869; C12Q 2600/156

(57) ABSTRACT

Systems and methods are provided for early intervention for PKD. One embodiment comprises a method for selectively recommending treatment for a patient for Polycystic Kidney Disease (PKD). The method includes reviewing sequencing data for the patient to determine if the patient has at least one qualifying variant in a gene PKD1 or a gene PKD2, the qualifying variant selected from the group consisting of Loss of Function (LoF) variants and coding variants. The method includes, if the patient does not have a qualifying variant in the gene PKD1 or the gene PKD2, generating a report recommending a first set of criteria for performing a PKD intervention. The method still further includes, if the patient does have a qualifying variant in the gene PKD1 or the gene PKD2, generating a report recommending a second set of criteria for performing the PKD intervention.

### RELATED APPLICATIONS

This application claims the benefit of U.S. Provisional Application Ser. No. 63/471,589, filed Jun. 7, 2023, entitled “Polycystic Kidney Disease Diagnosis and Treatment Based on Detection of PKD1/PKD2 Variants,” the disclosure of which is hereby incorporated by reference in its entirety.

### FIELD

The disclosure relates to the field of genomic analysis, and in particular, to treatment of patients relating to polycystic kidney disease.

### BACKGROUND

Chronic kidney disease (hereinafter, “kidney disease”) is a long-term, progressive disease that results in impaired functioning of the kidneys. Upon reaching an advanced stage, chronic kidney disease requires dialysis and/or an organ transplant. Polycystic Kidney Disease (PKD) is a form of kidney disease wherein cysts form that impair the functioning of the kidney.

Because of the notable and progressive lifestyle impact resulting from PKD, it remains critical to identify the presence of PKD for an individual patient as accurately as possible and as early as possible. Accurate diagnosis on a population scale is also critical, because one in one thousand. Americans are expected to develop PKD in their lifetime. That is, at least one half million Americans are expected to be impacted by this disease, and as such imprecision in detection of PKD will negatively impact the efficient use of critical medical resources on a nationwide scale.

Hence, scientists and medical practitioners continue to seek out enhanced systems and methods for detecting and treating PKD in a precise and accurate manner.

### SUMMARY

Embodiments described herein beneficially detect the presence of a qualifying genetic variant for the gene PKD1 and/or PKD2 within a patient, and use this information in combination with medical data (e.g., indicating hypertension, and/or above-normal levels of certain biomarkers) to detect and treat PKD before the onset of notable symptoms. Early treatment helps to extend life and quality of life for these patients who are more likely to experience PKD than their peers. Furthermore, because these processes are scalable for large populations (e.g., millions of people), they have the potential to greatly reduce both waste and underutilization of medical resources.

One embodiment is a method for selectively recommending treatment for a patient for Polycystic Kidney Disease (PKD). The method includes reviewing sequencing data for the patient to determine if the patient has at least one qualifying variant in a gene PKD1 or a gene PKD2, the qualifying variant selected from the group consisting of Loss of Function (LoF) variants and coding variants. The method includes, if the patient does not have a qualifying variant in the gene PKD1 or the gene PKD2, generating a report recommending a first set of criteria for performing a PKD intervention. The method still further includes, if the patient does have a qualifying variant in the gene PKD1 or the gene PKD2, generating a report recommending a second set of criteria for performing the PKD intervention.

A further embodiment is a method for selectively treating a patient for Polycystic Kidney Disease (PKD). The method includes determining whether the patient is genetically prone to development of kidney cysts, by: obtaining or having obtained a biological sample from the patient, and performing or having performed sequencing on the biological sample to determine if the patient has at least one qualifying variant in either a gene PKD1 or a gene PKD2, the qualifying variant selected from the group consisting of Loss of Function (LoF) variants and coding variants. The method also includes obtaining results of a blood pressure test upon the patient. If the results of the blood pressure test indicate hypertension for the patient and the patient has a qualifying variant in the gene PKD1 or the gene PKD2, the method further comprises triggering a PKD intervention. If the results of the blood pressure test indicate hypertension for the patient and the patient does not have a qualifying variant in the gene PKD1 or the gene PKD2, the method further comprises refraining from triggering the PKD intervention.

A further embodiment is a method for selectively treating a patient for Polycystic Kidney Disease (PKD). The method includes determining whether the patient is genetically prone to development of kidney cysts, by: obtaining or having obtained a biological sample from the patient, and performing or having performed sequencing on the biological sample to determine if the patient has at least one qualifying variant in either a gene PKD1 or a gene PKD2, the qualifying variant selected from the group consisting of Loss of Function (LoF) variants and coding variants. The method further comprises obtaining results of a biomarker measurement for the patient. If the results of the biomarker measurement meet a predefined criteria and the patient has a qualifying variant in the gene PKD1 or the gene PKD2, the method comprises triggering a PKD intervention. If the results of the biomarker measurement meet a predefined criteria and the patient does not have a qualifying variant in the gene PKD1 or the gene PKD2, the method comprises refraining from triggering the PKD intervention.

A further embodiment is a method for selectively treating a patient for Polycystic Kidney Disease (PKD). The method includes determining whether the patient is genetically prone to development of kidney cysts, by: obtaining or having obtained a biological sample from the patient; and performing or having performed sequencing on the biological sample to determine if the patient has at least one qualifying variant in either a gene PKD1 or a gene PKD2, the qualifying variant selected from the group consisting of Loss of Function (LoF) variants and coding variants. The method also includes obtaining results of a blood pressure test upon the patient. If the results of the blood pressure test indicate hypertension for the patient, then the method includes directing a biomarker determination for the patient, the biomarker measurement selected from a group consisting of a creatinine measurement, a urea measurement, a cystatin C measurement, and an estimated Glomerular Filtration Rate (eGFR) calculation from measurements taken from the patient, if the patient has a qualifying variant in the gene PKD1 or the gene PKD2. If the results of the blood pressure test indicate hypertension for the patient, then the method includes directing a biomarker measurement for the patient, then the method includes refraining from directing the biomarker measurement if the patient does not have a qualifying variant in the gene PKD1 or the gene PKD2.

Other illustrative embodiments (e.g., methods and computer-readable media relating to the foregoing embodiments) may be described below. The features, functions, and advantages that have been discussed can be achieved independently in various embodiments or may be combined in yet other embodiments, further details of which can be seen with reference to the following description and drawings.

### DESCRIPTION OF THE DRAWINGS

Some embodiments of the present disclosure are now described, by way of example only, and with reference to the accompanying drawings. The same reference number represents the same element or the same type of element on all drawings.

FIG. 1 is a diagram depicting a sample processing architecture in an illustrative embodiment.

FIG. 2 is a block diagram illustrating a genomics architecture in an illustrative embodiment.

FIGS. 3-4 are flowcharts depicting methods of providing alternative diagnostic and treatment thresholds for PKD, based on status as a carrier of a variant in the gene PKD1 and/or PKD2 and a medical measurement, in illustrative embodiments.

FIG. 5 is a table that summarizes biomarker threshold data in an illustrative embodiment.

FIG. 6 is a table that summarizes hypertension threshold data in an illustrative embodiment.

FIG. 7 is a table that summarizes biomarker test data for individuals in an illustrative embodiment.

FIG. 8 is a table that summarizes sequencing data for the genes PKD1 and PKD2 for individuals in an illustrative embodiment.

FIG. 9 is a table that summarizes variant data for the genes PKD1 and PKD2 for individuals in an illustrative embodiment.

FIGS. 10-11 depict Graphical User Interfaces (GUIs) that facilitate acquisition of PKD1/PKD2 carrier status, and/or tests for PKD diagnosis and treatment for a carrier of a PKD1/PKD2 variant, in illustrative embodiments.

FIG. 12 is a flowchart depicting a method of reporting different criteria for PKD diagnosis and intervention, based on status as a carrier of a PKD1/PKD2 variant, in an illustrative embodiment.

FIG. 13 is a flowchart depicting another method of providing alternative diagnostic and treatment thresholds for PKD, based on status as a carrier of a variant in the gene PKD1 and/or PKD2 and a medical measurement, in an illustrative embodiment.

FIG. 14 depicts an illustrative computing system operable to execute programmed instructions embodied on a computer readable medium.

### DESCRIPTION

The figures and the following description depict specific illustrative embodiments of the disclosure. It will thus be appreciated that those skilled in the art will be able to devise various arrangements that, although not explicitly described or shown herein, embody the principles of the disclosure and are included within the scope of the disclosure. Furthermore, any examples described herein are intended to aid in understanding the principles of the disclosure, and are to be construed as being without limitation to such specifically recited examples and conditions. As a result, the disclosure is not limited to the specific embodiments or examples described below, but by the claims and their equivalents.

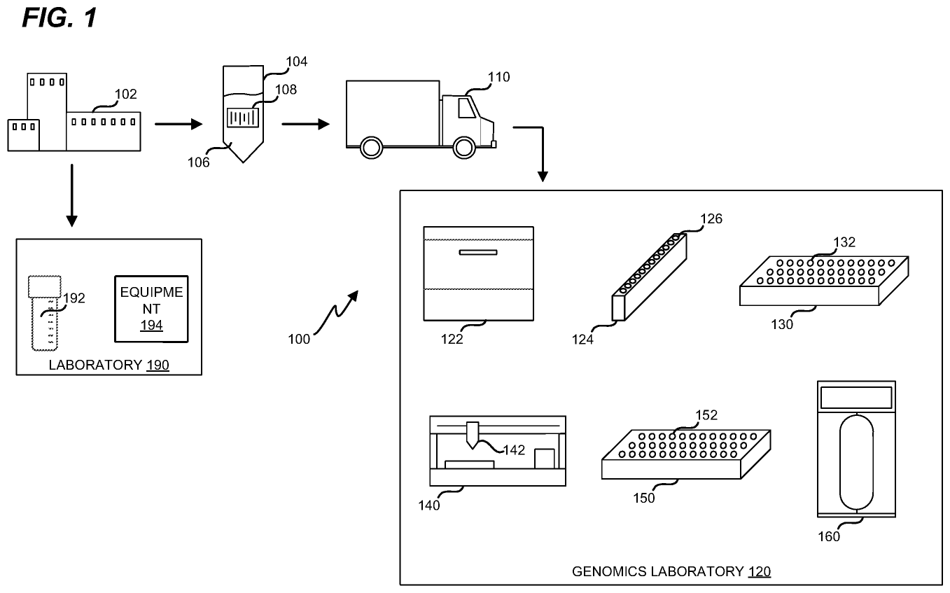

FIG. 1 is a diagram depicting a sample processing architecture 100 in an illustrative embodiment. Sample processing architecture 100 comprises any system or organizational structure for acquiring and sequencing biological samples in a high-volume, high-throughput manner. Sample processing architecture 100 may be utilized, for example, to collect and sequence genetic material (in the form of Ribonucleic Acid (RNA) or Deoxyribonucleic Acid (DNA)) found within thousands or tens of thousands of samples 106 daily, via multiple healthcare provider networks 102.

Healthcare provider networks 102 may comprise hospitals, clinics, practitioner offices, laboratories, surgical centers, etc. that engage in or facilitate the practice of medicine. In one embodiment, healthcare provider networks 102 each comprise groups of hospitals that treat millions of patients. As a part of the practice of medicine, healthcare provider networks 102 acquire samples 106 for sequencing. For example, a healthcare provider network 102 may acquire samples 106 as part of a population screening program, as part of medical treatment, etc. The specific amount of sequencing desired for a sample 106 may comprise a selected set of one or more genes, an exome, the entire genome of a patient, etc. The samples 106 are stored in sample containers 104, which may be accompanied by Customer Sample Identifiers (CSIs) 108. A delivery service 110 provides the samples 106 to a genomics laboratory 120 for processing.

Healthcare provider networks 102 may also acquire samples 192 for blood testing (described below). These samples 192 may be provided to laboratory 190 for analysis via equipment 194 (e.g., a chemically treated test strip, biochemical assay, etc.), or may be analyzed by patients via at-home testing methods. Sample processing architecture 100 provides a technical benefit by allowing laboratory 190 and genomics laboratory 120 to specialize in different methods of analysis.

Procedures within genomics laboratory 120 related to genetics may include accessioning, sample plating, storage, extraction, library preparation, enrichment, and sequencing processes. These processes acquire genetic material from a sample 106, separate the genetic material from other constituents, duplicate the genetic material, and quantify the genetic material order to determine a swathe of sequence data, such as an exome or entire genome for a subject (e.g., a human patient, an organelle of a human patient, etc.). Although the procedures discussed herein are specific with regard to one method of sequencing, other techniques may be utilized in accordance with known standards in order to perform sequencing for samples 106. For example, although the techniques discussed herein relate to hybridization capture techniques, amplicon-based techniques may be used.

### Accessioning

Accessioning refers to receiving and preparing samples 106 for later laboratory processes. In one embodiment, accessioning includes receiving a batch of samples 106 (e.g., hundreds or thousands of samples 106) from one or more delivery services 110 each day for processing. For example, packages that each include tens or hundreds of samples 106 may be delivered to genomics laboratory 120 via the United States Postal Service (USPS), or a private package carrier.

Each sample 106 may be retained within a sample container 104, such as a five milliliter (mL) test tube. In this embodiment, the sample container 104 is sealed to prevent the sample 106 from being exposed to the environment and also to prevent the sample 106 from co-mingling with other samples 106. For example, the sample 106 may be sealed via a cap that is threaded, glued, press-fit, etc. At the time of delivery, the sample container 104 may further include a remnant of a sampling tool, such as a portion of a swab that was utilized to acquire the sample.

In many embodiments, a CSI 108 for the sample 106 is reported via a component affixed to or integrated with the sample container 104. The CSI 108 uniquely distinguishes the sample 106 from other samples 106 being received. For example, a CSI 108 may uniquely distinguish a sample 106 from other samples 106 in the same batch, other samples 106 received on the same date, other samples 106 received from the same healthcare provider network 102, etc. A CSI 108 may be reported via a barcode label, Quick Response (QR) code label, Radio Frequency Identifier (RFID) chip, or any suitable visual, transmission-generating, or other physical component affixed to or integrated with the sample container 104.

In further embodiments, the sample container 104 is itself sealed within an external container such as a bag (not shown). Using an external container helps to prevent contamination, by ensuring that a technician at the genomics laboratory 120 does not contact biological material from the sample 106 that may exist on an outer surface of the sample container 104. Use of an external container may also be required by law (e.g., Department of Transportation (DOT) guidelines). Use of an external container additionally helps to prevent cross-contamination between samples 106. Furthermore, in embodiments where samples 106 may include blood or a pathogen, an external container provides an additional barrier to protect the health of technicians. The external container may additionally include documentation confirming the CSI 108, information for the subject that the sample was sourced from, and/or information indicating circumstances of sampling. The circumstances of sampling may include, for example, a sampling date, a sampling method, a location that the sample was acquired, a name or title for a person who performed the sampling, and/or additional notes.

In this embodiment, the sample 106 comprises a chemical solution. For example, the sample 106 may comprise a prepared aqueous solution such as a saline solution, or may comprise a bodily fluid such as blood, saliva, mucus, etc. In some embodiments each of the samples 106 fills between two and five milliliters of volume within its corresponding sample container 104.

The samples 106 further include genetic material such as Deoxyribonucleic Acid (DNA), Ribonucleic Acid (RNA), etc. In many instances, the genetic material is one of many constituent components within the sample 106. For example, the genetic material may exist within the nuclei of white blood cells that are included within the sample 106. In a further example, genetic material may exist within viruses or bacteria within the sample 106. In this embodiment, the genetic material is not yet isolated from the remaining constituent components of the sample 106.

After receipt of the samples 106, batches of the samples 106 (e.g., as stored within sample containers 104 and/or external containers) may be heated in ovens 122 to facilitate cell lysis. The temperature, and duration of heating, may be chosen such that pathogenic material within the samples 106 is rendered harmless, or such that cellular lysis occurs. For example, heating may occur at a temperature of between forty and eighty (e.g., fifty) degrees Celsius (C), for a period of time between fifteen and two hundred (e.g., thirty) minutes. In some embodiments, including embodiments wherein the samples 106 are primarily the contents of a blood draw, the heating step may be foregone.

In this embodiment, upon completion of heating, the batches of samples 106 are removed from the ovens 122. In one embodiment, sample containers 104 are removed from corresponding external containers, such as by cutting the external containers open. With the sample containers 104 now available for direct interaction, the sample containers 104 are inspected. As a part of this process, a technician or automated system may determine the CSI 108 for the sample 106, and may compare the CSI 108 to a CSI 108 listed on documentation provided in the external container. If there is a discrepancy between the CSI 108 on the sample container 104 and a CSI 108 listed in the documentation, the sample 106 may be flagged as having an error condition. Similarly, if the CSI 108 on the sample container 104 is damaged (e.g., abraded, heat-damaged, or water-damaged) and has become unreadable, the sample 106 may be flagged as having an error condition.

A technician or automated system may further inspect the contents of the sample container 104, via visual or other methods. If the sample 106 does not include an expected constituent component (or is otherwise non-compliant) then the sample 106 is flagged as having an error condition. For example, if the sample 106 is primarily saliva and includes a fluid that is not permitted (e.g., blood), includes an entire swab or no swab, appears to have a fractured or broken casing, or is outside of an expected range of volume (e.g., between two and five milliliters), then the sample 106 may be flagged as having an error condition.

Samples 106 that have not been flagged as having an error condition proceed to sample integration. In one embodiment, as a part of sample integration, the sample 106 is assigned a Laboratory Sample Identifier (LSI). The LSI uniquely identifies the sample 106 from other samples 106 received for the batch, received on the same day, processed in the same laboratory, and/or handled by the same organization performing sequencing. In many embodiments, the LSI is stored in a memory of a genomics server (e.g., within a laboratory sample database), and is uniquely associated with a corresponding CSI 108 for the sample. The LSI may also be associated with any error conditions reported for the sample 106.

In many embodiments, CSIs 108 originally provided with the samples 106 are in the form of a paper barcode. In such embodiments, the paper barcode may be printed in aqueous ink. This renders the barcode subject to degradation upon exposure to liquid in the laboratory environment, which is undesirable.

To ensure that each sample container 104 is capable of traveling through the genomics laboratory 120 without its identifier being physically degraded, a corresponding LSI may be indicated at the sample container 104. The LSI may be indicated via the application of a barcode label, Quick Response (QR) code, Radio Frequency Identifier (RFID) chip, or other visual, transmission-generating, or other physical component affixed to or integrated with the sample container.

In one embodiment, the LSI is printed onto a barcode label comprising rip-proof material (e.g., vinyl) in a water-insoluble ink. This implementation ensures that the barcode label is resistant to physical and chemical degradation. The barcode may be applied around an entire perimeter of the sample container 104, ensuring that the sample container 104 may be scanned from any angle.

In further embodiments, the element used to report the LSI is accompanied by a visually distinct mark that enables rapid confirmation by a technician that the sample 106 has been integrated into the laboratory environment. The visually distinct mark may comprise a colored ring (e.g., around an entire perimeter of the sample container), a logo, a physical feature, a stamp, etc.

### Sample Plating

With the samples 106 having been successfully integrated into the environment of the genomics laboratory 120 environment, the samples 106 are ready for analytics to be performed. To this end, the samples 106 are prepared for transfer to a sample microplate 130. The sample microplate 130 may be labeled with a unique identifier via similar techniques to those used for sample containers 104 above. The unique identifier distinguishes the sample microplate 130 from other sample microplates 130. In one embodiment, the sample microplate 130 comprises a solid body defining three hundred and eighty-four wells, distributed across sixteen rows and twenty-four columns, each well having a capacity of between thirty and one hundred microliters. In a further embodiment, the sample microplate 130 comprises a solid body defining ninety-six wells, distributed across eight rows and twelve columns, each well having a capacity of between one hundred and three hundred microliters. Any suitable number and arrangement of wells may be selected as a matter of design choice.

As a part of preparing the samples 106 for transfer to the sample microplate 130, a technician may place sample containers 104 onto a rack 124, and scan each sample container 104 to determine an LSI for each location 126 (e.g., each container receptacle) on the rack 124. In some embodiments, the rack 124 is assigned a unique identifier that distinguishes it from other racks 124. The rack 124 may be labeled with a unique identifier using techniques similar to those used for sample containers 104. The technician, or automated machinery such as a server operating an optical scanner, may then associate the unique identifier for the rack 124, along with the locations 126 assigned to the samples 106, with the corresponding LSIs of the samples 106 stored at the rack 124.

The technician additionally unseals the sample containers 104. Unsealing of sample containers 104 may be a deeply labor-intensive process, particularly when laboratory processes are performed at scale to handle tens of thousands of samples 106 per day. Thus, a technician may utilize automated tooling to enhance the speed at which sample containers 104 are unsealed. The tooling may, for example, unscrew, cut, or drill each sample container 104, in order to make the sample 106 within available for physical transfer to the sample microplate 130.

One or more racks 124 of samples 106 are provided to a Liquid Handler (LH) 140, such as an automated robot that operates an end effector 142 in accordance with one or more Numerical Control (NC) programs to transfer liquids between wells via arrays of micropipettes. An LH 140 is also known as a “Liquid Handling System.” LH 140 may comprise, for example, a Hamilton Microlab Star Liquid Handling System.

In this embodiment, the LH 140 proceeds to transfer a portion of each sample 106 at a rack 124 to a well 132 within the sample microplate 130 that is not shared with other samples 106. For example, the well 132 for each sample 106 may be predetermined in accordance with a control program used by the genomics laboratory 120. In one embodiment, the LH 140 transfers the portions of the samples 106 to the wells 132 of the sample microplate 130 by providing instructions to actuators, piezoelectric elements, and/or pressure systems operating the end effector 142. In such an embodiment, the end effector 142 may align its array of micropipettes with the sample containers 104 to retrieve portions of the samples 106. Furthermore, in such an embodiment, the end effector 142 may dynamically align its array of micropipettes with the sample microplate 130 to deposit the portions of the samples 106 at the wells 132.

Because there is a known relationship between locations 126 at the rack 124 and wells 132 of the sample microplate 130 (e.g., as indicated by row and column), contents of the memory of a genomics server (e.g., a laboratory sample database) may be updated to indicate the well 132 storing genetic material for each sample 106. In one embodiment, the memory is further updated to associate a unique identifier for the sample microplate 130 with the samples 106 stored therein.

In one embodiment, programmed instructions for the LH 140 may direct the end effector 142 to position itself above a set of disposable tips, descend into the tips to attach the tips, reposition the end effector 142 above the rack of sample containers 104, adjust spacing between micropipettes within the array, descend until the tips reach the sample containers 104, draw liquid from the sample containers 104, deposit the liquid into a well at the sample microplate 130, and then dispose of the tips. Such a process may be repeated across sample containers 104 stored on multiple racks until the sample microplate 130 is filled with portions from the samples 106. In one embodiment, one or more wells 132 on the sample microplate 130 are filled with a control reagent instead of a portion of a sample 106.

The amount of liquid drawn from each sample container 104 may comprise a small fraction of the overall volume of the sample container 104. For example, an amount of liquid drawn may comprise several microliters, such as between two and ten microliters. Upon completion of transfer from the sample containers 104 to the wells, the sample microplate 130 may be covered with a liquid and/or gas-impermeable layer, such as foil or paraffin. Sample containers 104 remaining on the racks may be resealed, for example with pressure-fit caps having a color distinct from an original color for the sample containers. With accessioning now complete for the sample microplate 130, the sample microplate 130 is transferred to a next section of the laboratory for processing.

### Storage

In one embodiment, accessioned samples 106, samples 106 ready for analytics, and/or samples 106 that have already been sequenced, are stored for later use. For example, samples 106, sample containers 104, and/or sample microplates 130 may be stored at room temperature, or may be cryogenically frozen at a low temperature (e.g., negative eighty degrees Celsius) and arranged in racks for later retrieval. Samples 106 may be preserved for periods of days or years, enabling rapid re-testing to be performed for subjects without the need for re-acquiring genetic material. Storage of the samples 106 provides notable value in the event that contents of a well 132 used for sequencing do not meet with rigorous quality control standards.

Specifically, storage enables re-sampling to occur in the event that there is a desire to re-sequence a sample 106.

### Extraction

Sample microplates 130 are transferred to a portion of the genomics laboratory 120 dedicated to extraction of the genetic material. The segment of the laboratory 120 that performs extraction and other pre-amplification operations may be sealed from, and/or positively pressurized relative to, other portions of the genomics laboratory 120.

During extraction, a sample microplate 130 is acquired and provided to an LH 140. The LH 140 that performs extraction may be different from the LH 140 that performs sample plating. The LH 140 may apply a reagent to each well 132 that lyses cells within each well. For example, this may be performed in order to lyse white blood cells containing genetic material for a human, or may comprise lysing other types of cells to expose other types of genetic material. The reagents used for pre-amplification processes may be stored at the LH 140 in a temperature-controlled manner, and may even be vibrated or mixed on a regular basis to ensure that the reagents are evenly distributed in suspension.

In one embodiment, extraction further includes an LH 140 aspirating and dispensing reagents that selectively bind to genetic material released from the lysed cells. This process may include applying a bead (not shown) to the well 132. In one embodiment, the beads comprise magnetic beads that selectively bind to the genetic material (e.g., DNA). This allows for isolation and purification of the genetic material while contaminants remain in solution. In one embodiment, the magnetic bead is drawn to a magnetic base at or under the sample microplate 130. After the genetic material has been drawn to the bead, and after the bead has been secured to the base of the well, a flushing step may be performed wherein remaining fluid in each well is washed away. This ensures that potential impurities are removed from the well. The LH 140 may further add or remove fluid from each well 132 to perform additional concentration and/or elution of the genetic material, and may transfer fluid from the wells 132 of the sample microplate 130 to wells 152 of a genome stock microplate 150. The genome stock microplate 150 may be labeled with a unique identifier, and the contents of each well 152 of the genome stock microplate 150 may be associated with a corresponding LSI. In all phases of operation, the LH 140 is operated to ensure that fluid is not transferred between wells 152, as this results in contamination.

In one embodiment, a portion of fluid is removed from each well 152 of the genome stock microplate 150 for quality control purposes. Concentration of genetic material within the wells 152 may be confirmed via testing of this fluid, such as by application of a dye that reacts with the genetic material at known levels of fluorescence for known concentrations.

### Library Preparation

After extraction is completed, library preparation may be performed for the contents of the genome stock microplate 150. The bead for each well, including ionically bonded genetic material, is transferred to a distinct well of a library preparation microplate (not shown). The library preparation microplate includes an identifier that uniquely distinguishes it from other library preparation microplates, and the LSI associated with each well on the genome stock microplate 150 may be mapped to a corresponding well on the library preparation microplate.

The library preparation microplate may be transferred to a new portion of the genomics laboratory 120 that is sealed from, and/or positively pressurized relative to, other portions of the genomics laboratory 120 that do not perform amplification of genetic material. This feature helps to prevent amplified genetic material from entering portions of the laboratory where genetic material has not been amplified, which could result in contamination. The transfer process may be performed by placing a library preparation microplate into an airlock at the pre-amplification portion of the genomics laboratory 120, sealing the airlock, and then retrieving the library preparation microplate from the airlock via the amplification portion of the genomics laboratory 120.

In one embodiment, a reagent is applied to each well of the library preparation microplate. The reagent ionically bonds to the surface of the bead within the well, and does so more strongly than the genetic material. This releases the genetic material from the surface of the bead of each well, enabling the genetic material to be chemically interacted with.

Library preparation may include normalization of a concentration of genetic material in each well of the library preparation microplate. Library preparation further includes fragmentation of the genetic material via an enzyme or via the application of physical forces. During this process, the entire genome (e.g., roughly three billion base pairs for a human genome), may be fragmented into pieces. In one embodiment, the pieces vary between three hundred and four hundred base pairs in length. These pieces are known as nucleic acid fragments.

In this embodiment, the nucleic acid fragments undergo adaptor ligation and indexing in accordance with known techniques. For example, this may comprise Next Generation Sequencing (NGS) library preparation processes defined by Illumina. Next, a limited amount of Polymerase Chain Reaction (PCR) amplification is performed upon the library. The resulting solution is then purified and eluted via operation of an LH 140.

During library preparation, one or more reference samples of genetic material, distinct from the genetic material found in the samples, may be added to wells of the library preparation microplate. The reference samples do not include genetic material received from a customer, but rather include known sequences of base pairs. The reference samples serve as controls to ensure that processes are carried out with sufficient quality.

Upon completion of library preparation, desired fragments of the genetic material (e.g., thousands or millions of distinct fragments of the genetic material, each corresponding with a different portion of a genome of the subject) have been ligated to predefined adapters (e.g., DNA adapters) that bind with the genetic material. Each of the adaptor-ligated fragments is referred to as a “library.”

In further embodiments, the probes applied to each well of the library preparation plate include chemical identifiers (colloquially referred to as “barcodes”) that are distinct from each other. The use of a different chemical identifier for probes applied to each well of the library preparation microplate enables sequencing to later be performed for multiple subjects on the same flow cell, without conflating sequencing results for those subjects.

The library preparation process may further comprise controlling a concentration of the genetic material in each well, and purification and/or elution of the resulting material. Similar to the processes performed after extraction of genetic material, concentration of genetic material after library preparation may be confirmed for each well via testing.

### Enrichment

After library preparation, enrichment processes may be performed in order to either directly amplify (e.g., via amplicon or multiplexed PCR) or capture (e.g., via hybrid capture) predefined libraries. This enhances the ease of sequencing desired portions of the genome.

In one embodiment, during enrichment, customized biotinylated oligonucleotide probes are applied to the libraries. The probes selectively hybridize genetic material occupying desired portions of the genome for the genetic material, such as specific genes, or the entire exome. Magnetic beads bind to biotin molecules in the probes to attach the hybridized material to the magnetic beads. Magnetic forces capture the beads in place, enabling remaining fluid within each well to be removed or washed out, thereby removing impurities and leaving only the genetic material that is desired. Genetic material may be released from the beads in a similar manner to that discussed above for prior processes.

In a further embodiment, hybrid capture target enrichment is performed. During this process, the probes comprise tailored oligonucleotides that are chosen to bind to the genetic material. The range of probes may be tailored as a group to bind to specific alleles, specific genes, the exome, the entire genome, etc. That is, each probe may bind to a nucleic acid fragment at a specific location on the genome, and the range of probes may be selected to ensure that alleles, genes, the exome, or the entire genome of the subject being considered is acquired. Utilizing probes in this manner may enhance efficiency of the sequencing process, by foregoing the need to sequence all of the roughly three billion base pairs found in the human genome.

The enrichment process may further comprise controlling a concentration of the genetic material in each well, and purification and/or elution of the resulting material. Similar to the processes performed after extraction of genetic material, concentration of genetic material after enrichment may be confirmed for each well via testing.

### Sequencing

Sequencing may be performed according to any of a variety of techniques, including short-read and long-read techniques, via sequencing equipment 160 (e.g., an Illumina NovaSeq X sequencing machine). In one embodiment, the sequencing is performed as Sequencing by Synthesis (SBS). For example, sets of enriched libraries of genetic material bound to probes in earlier steps may be transferred to a flow cell, and annealed to oligonucleotide probes within the flow cell. At this stage, the contents of multiple wells may be applied to the same flow cell, because the libraries within those wells are tagged with the chemical identifiers referred to above. In one embodiment, the chemical identifiers comprise nucleotide sequences that are detectable during the sequencing process to determine a corresponding LSI.

Complementary sequences may then be created via enzymatic extension to create a double-stranded portion of genetic material. The double-stranded genetic material may then be denatured, and the library fragment may be washed away. Bridge amplification may then be performed to create copies of the remaining molecule in a localized cluster. For example, a cluster may comprise twenty to fifty copies of the same molecule, localized to a location the size smaller than a pinhead on the flow cell.

In this embodiment, sequencing primers are annealed to library adapters in order to prepare the flow cell for SBS. During SBS, the sequencing primer uses reverse terminator fluorescent oligonucleotides, one base per cycle, for a number of cycles (e.g., one hundred and fifty cycles) in the forward direction. After the addition of each nucleotide, clusters are excited by a light source, resulting in fluorescence which can be measured. The emission wavelength and signal intensity for each cluster determines a base call for that cluster. Fluorescent moieties are then flushed from the flow cell. A chemical group blocking a 3′ end of the fragment is then removed, enabling a subsequent nucleotide to be read. This tightly controls nucleotide addition and detection.

Additionally in this embodiment, base calls across cycles at the same physical location on the flow cell occur at the same cluster, and hence indicate sequential reads for copies of the same fragment of the genetic material. After each cycle, denaturing and annealing are performed to extend the index primer. A complementary reverse strand is created and extended via bridge amplification. The reverse strand is then read in the reverse direction for a number of cycles, in a manner similar to reads in the forward direction.

Depending on whether a complete human genome, or another set of genomic data, is being tested, different reagents (e.g., probes, primers, etc.) may be chosen. That is, different reagents may be utilized for library preparation for a pathogen (e.g., bacteria, virus) or an organelle (e.g., mitochondria) than for a human genome. Pathogens exhibiting Ribonucleic Acid (RNA) genomes may have their genetic material translated to DNA before sequencing, enrichment, and/or library preparation are performed, via known techniques, such as Next Generation Sequencing (NGS) techniques.

Throughout the processes discussed above, the laboratory environment may be carefully controlled to ensure quality. For example, temperature within each segment of the laboratory may be carefully monitored and controlled, and ultraviolet lighting or other features capable of inactivating genetic material may be carefully positioned to ensure that contamination does not occur.

### Bioinformatics

Sequencing data may be stored in any suitable format. In one embodiment, raw sequencing data generated during synthesis is stored in a file format such as Binary Base Call (BCL). This raw data may be fed to an analytical pipeline such as a cloud-based computing environment. Raw sequencing data may be processed by the pipeline into a second format, such as a text-based FASTQ format, that reports quality scores. The second format may then analyzed to perform alignment of sequence reads to a reference genome, such as a reference genome reported in a Browser Extensible Data (BED) file. The aligned sequence data may be reported as a Binary Alignment Map (BAM) file or Compressed Reference-oriented Alignment Map (CRAM) file. The aligned sequence data may then be called, resulting in a Variant Call Format (VCF) file reporting called variants at each location of the genome that was sequenced, together with secondary metrics such as quality indicator metrics. As used herein, a variant comprises a unique combination of genetic information, in the form of consecutive base pairs at a specific set of locations (e.g., genomic coordinates) along a portion of a chromosome. Each variant is distinguished from other variants by having a different combination of base pairs along the set of locations. This may be due to Single Nucleotide Polymorphisms (SNPs) which relate to common single nucleotide changes, Single Nucleotide Variants (SNVs) which relate to rare nucleotide changes, insertions and/or deletions (Indels) which relate for example to the insertion or deletion of less than thirty base pairs, or differing numbers of repetitions, Copy Number Variants (CNVs), which relate to larger insertions or deletions, translocations, inversions, other types of genetic variants, or even combinations of variants, such as haplotypes or Multi-nucleotide variants (MNVs).

The called sequence data may be provided to a data analyst via a User Interface (UI), such as a Graphical User Interface (GUI) presented via a display. The technician may then validate the resulting called sequence data and release it for reporting to subjects, health care providers, and/or scientists.

### Genomics Architecture

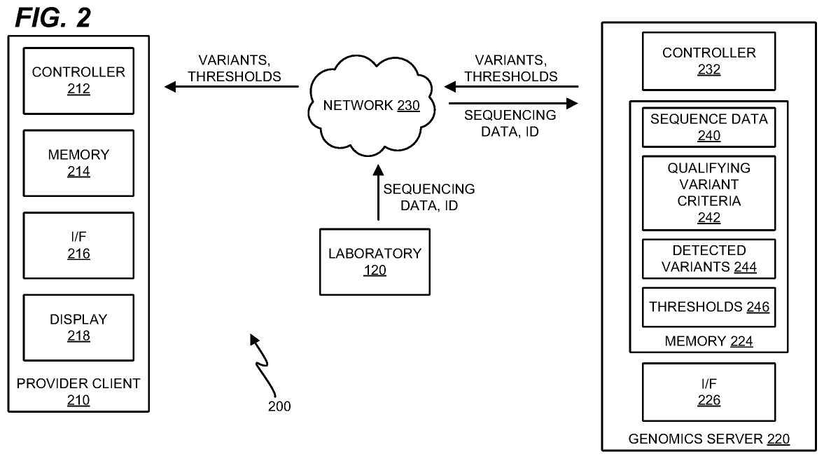

FIG. 2 is a block diagram illustrating a genomics architecture 200 in an illustrative embodiment. Genomics architecture 200 comprises any combination of systems and devices operable to review, process, and/or control access to sequencing data, including sequencing data received from genomics laboratory 120. In this embodiment, genomics architecture 200 comprises a genomics server 220 which receives sequencing data and identifiers (e.g., CSIs 108, LSIs, etc.) from genomics laboratory 120, via network 230.

Genomics server 220 receives the sequencing data via interface (I/F) 226, such as an Ethernet interface, wireless interface compliant with Institute of Electrical and Electronics Engineers (IEEE) 802.11 standards, or other physical interface capable of transmitting and receiving digital data. The sequencing data 240 is stored in memory 224 for the population of patients (e.g., millions of patients) that have been sequenced by laboratory 120, and may be maintained in any suitable format. Examples of such formats include CRAM, VCF, BAM, and others. Memory 224 may store, for example, sequence data 240 describing multiple patients, and this sequence data 240 may be maintained in a de-identified format to facilitate the advancement of research. Memory 224 may be implemented via a cloud storage service, or may comprise a storage medium such as a hard disk or flash memory device.

Memory 224 additionally stores qualifying variant criteria 242, detected variants 244, and thresholds 246 for diagnosis and/or treatment of Polycystic Kidney Disease (PKD). In one embodiment, the portion of memory 224 storing these components is distinct from the portion of memory 224 storing sequence data 240.

Controller 232 manages the operations of genomics server 220, and may for example analyze sequence data 240 to identify detected variants 244, control access and authentication related to sequence data 240, communicate with one or more provider clients 210, and/or perform additional operations. Controller 232 may be implemented, for example, as custom circuitry, as a hardware processor executing programmed instructions, as a combination of shared hardware processing resources implementing a compute service, or some combination thereof.

Genomics architecture 200 further comprises provider client 210, which is configured to receive information regarding detected variants 244 and/or thresholds 246. In this embodiment, provider client 210 includes a controller 212, a memory 214, an interface (I/F) 216, and a display 218. Controller 212 manages the operations of the provider client 210, and may be implemented, for example, as custom circuitry, as a hardware processor executing programmed instructions, or some combination thereof. Memory 214 comprises information for interpreting the data received via I/F 216. Display 218 may comprise a projector, screen, etc. for presenting information to a user of provider client 210.

### Interpreting PKD1 and PKD2 Sequencing Data

After sequencing data for the patient has been acquired (e.g., as an accompaniment to blood testing, in a prior event that provided a sample 106, etc.), sequencing data for the gene PKD1 and for the gene PKD2 is reviewed for the patient by controller 232 of genomics server 220. PKD1 is the gene that codes for polycystin-1. PKD1 has a cytogenic location of 16p13.3, and genomic coordinates of (GRCh38): 16:2,088,708-2,135,898. PKD2 is the gene that codes for polycystin-2. PKD2 has a cytogenic location of 4q22.1, and genomic coordinates of (GRCh38): 4:88,007,635-88,077,777). The sequencing data may further be used with a Minor Allele Frequency (MAF) cutoff (as discussed below), to prevent common variants from being analyzed.

In one embodiment, reviewing sequencing data for the patient comprises inspecting VCF data within the genomic coordinates for the gene PKD1 and the gene PKD2, and using a tool such as the Ensembl Variant Effect Predictor (VEP) to determine whether any called variants are expected to inactivate polycystin-1 or polycystin-2. These are referred to as “Loss of Function” (LoF) variants. LoF variants may include base pairs that indicate stop_lost, start_lost, splice_donor_variant, frameshift_variant, splice_acceptor_variant, or stop_gained. Such variants may include frame shift mutations, nonsense mutations, mutations at splice sites, insertions and/or deletions that result in stop codons, and others. Reviewing sequencing data for the patient may further comprise inspecting VCF data, and using the Ensembl VEP to identify coding variants within the genes being considered (i.e., PKD1 and PKD2). Coding variants comprise mutations that alter an amino acid coded for by the genes being considered, but do not inactivate glucokinase. For example, coding variants may include base pairs, residing in predetermined portions of the genes being considered, that indicate stop_lost, mis-sense_variant, start_lost, splice_donor_variant, inframe_deletion, frameshift_variant, splice_acceptor_variant, stop_gained, or inframe_insertion. Collectively, LoF variants and coding variants for the genes being considered are referred to as “qualifying variants.” In one embodiment, qualifying variants do not include Polyphen benign or Sorting Intolerant From Tolerant (SIFT) benign variants. Polyphen benign variants may be considered any variants having a Polyphen value less than 0.15, while SIFT benign may be considered any variants having a SIFT value that is greater than 0.05. In a further embodiment, variants having other predicted molecular properties, such as splice site variants, etc. are considered qualifying variants. The combination of criteria used to classify a variant as a qualifying variant is maintained in qualifying variant criteria 242, which is stored in memory 224.

The review process for detecting qualifying variants in the gene PKD1 and PKD2 for each patient may be performed automatically by an analytical tool to classify the patient as having an LoF variant, coding variant, or neither in these genes. For example, qualifying variants may be defined as including LoF variants having a MAF below 0.1%, and further including coding variants that are predicted as damaging by either SIFT or PolyPhen, wherein there is an agreement between a functional screen for PKD1 and PKD2 variants (as published in literature, such as “A comprehensive map of human glucokinase variant activity,” by Sarah Gersing et al. (bioRxiv, May 4, 2022)) and a moving-window analysis result for those variants (as discussed in U.S. patent application Ser. No. 17/575,894).

In one embodiment, the patient is classified as having a qualifying variant, or as not having a qualifying variant, whenever qualifying variants are detected at a confidence level of ninety-five percent or higher.

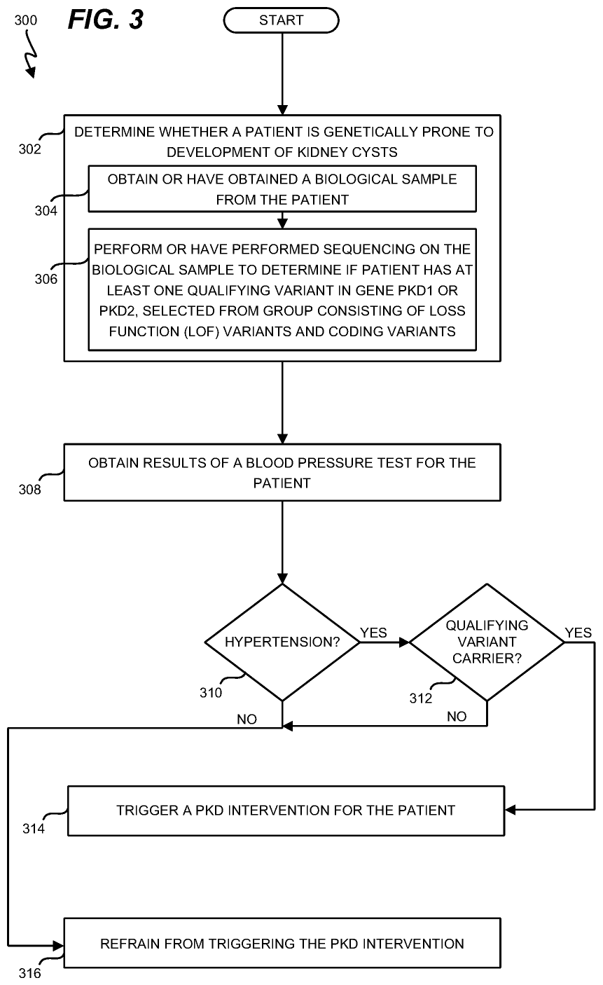

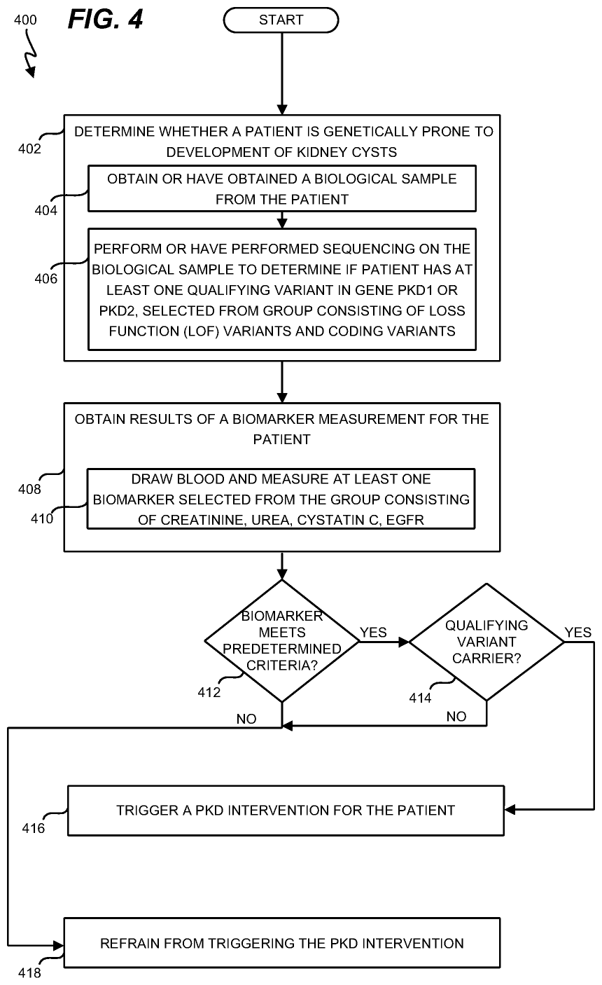

FIGS. 3-4 are flowcharts depicting methods of providing alternative diagnostic and/or treatment thresholds for PKD, based on status as a carrier of a variant in the gene PKD1 and/or PKD2 and a medical measurement, in an illustrative embodiment.

The steps of the methods herein are described with reference to sample processing architecture 100 of FIG. 1 and genomics architecture 200 of FIG. 2, but those skilled in the art will appreciate that these methods may be performed in other systems. The steps of the flowcharts described herein are not all inclusive and may include other steps not shown. The steps described herein may also be performed in an alternative order.

FIG. 3 provides a method 300 of selectively performing a PKD intervention upon a patient, based on a combination of qualifying variant carrier status and a medical measurement (e.g., as measured in an EHR) in an illustrative embodiment. Specifically, method 300 focuses on the concept of selectively performing PKD interventions based on medical data in the form of a blood pressure measurement. Step 302 comprises determining whether the patient is genetically prone to development of kidney cysts. In one embodiment, this comprises step 304 of obtaining (or having obtained) a biological sample from the patient, such as a sample 106 consisting essentially of blood or saliva. Step 306 includes performing (or having performed) sequencing on the biological sample to determine if the patient has at least one qualifying variant in the gene PKD1 or the gene PKD2 (e.g., via genomics server 220). The qualifying variant is selected from the group consisting of LoF variants and coding variants in PKD1 and PKD2. Steps 304-306 may be performed via the intake, accessioning, genomics laboratory, and bioinformatics processes discussed above with reference to FIG. 1, or via any suitable sequencing technique. In one embodiment, a determination that a patient is a carrier of a qualifying variant in PKD1 or PKD2 is made whenever a qualifying variant is called in a VCF file or similar data structure. In a further embodiment, this determination is made whenever a qualifying variant is confirmed by a variant scientist or automated system.

Step 308 includes obtaining results of a blood pressure test on the patient. This may comprise analyzing an EHR for the patient to determine results of a prior blood pressure test, or actively performing (or having performed) a blood pressure test upon the patient, and then updating the EHR of the patient. The blood pressure test may comprise application of a pressure cuff to the patient, followed by measuring systolic and/or diastolic blood pressure for the patient via use of the cuff. In further embodiments, dedicated sensors are used to determine blood pressure. After the EHR of the patient has been updated with the results, it may be read by genomics server 220 or provider client 210 in order to obtain the results. In a further embodiment, results older than a threshold age (e.g., one year, six months, three months, etc.) are not considered during this process, or are ignored if more recent results are available.

Step 310 comprises determining, based on the blood pressure test for the patient, whether the patient has hypertension. In one embodiment, this comprises genomics server 220 accessing an EHR of the patient for evidence of hypertension, as indicated by a code in a medical vocabulary. For example, an International Classification of Diseases, Tenth Edition (ICD-10) code of 10.9, or a Current Procedural Terminology (CPT) code of 99473 or 99474 may indicate the presence of hypertension as measured by the blood pressure test. In a further embodiment, step 310 comprises actively reviewing blood pressure measurements for evidence of hypertension. In the event that the patient has grade 1 hypertension (e.g., as indicated by a systolic pressure of 130-139 millimeters of mercury (mm Hg) and/or diastolic pressure of 80-89 mm Hg), or grade 2 hypertension (e.g., as indicated by a systolic pressure of 140 mm Hg or more, and/or a diastolic pressure of greater than 90 mm Hg), processing continues to step 312. In some embodiments, the blood pressure test is performed before the sequencing, and results of the blood pressure test are stored in the EHR for the patient.

Alternatively, if the patient does not have hypertension in step 310, then processing continues to step 316, and no PKD intervention is performed for the patient (e.g., unless PKD is later directly confirmed via medical imaging).

In step 312, genomics server 220 determines whether the patient is a carrier of a qualifying variant in PKD1, or a qualifying variant in PKD2 (e.g., according to the techniques and standards described above). In the event that the patient is a carrier of a qualifying variant, then processing proceeds to step 314.

Step 314 comprises performing a PKD intervention for the patient. A PKD intervention is an intervention designed to reduce or delay the negative long-term health outcomes associated with PKD. A PKD intervention may comprise prescribing angiotensin-converting enzyme (ACE) inhibitors or angiotensin II receptor blockers or other medications that lower blood pressure, diuretics, erythropoietin, statins or other drugs that lower cholesterol, tolvaptan, calcium or vitamin D supplements, and/or implementing a protein-reduced diet (i.e., a diet that permits consumption of no more than one hundred and eighteen grams of protein per day).

Method 300 provides a technical benefit over prior systems and techniques because it enables those who are genetically prone to PKD to be identified and treated prior to onset of life-altering symptoms, which in turn extends both the quantity and quality of life for such patients.

FIG. 4 provides a method 400 of selectively performing a PKD intervention upon a patient, based on a combination of qualifying variant carrier status and a medical measurement (e.g., as measured in an EHR) in an illustrative embodiment. Specifically, method 400 focuses on the concept of selectively performing PKD interventions based on medical data describing biomarkers found within bodily fluid (e.g., blood samples or urine samples). Steps 402-406 may be performed in a similar manner to steps 302-306 of method 300 of FIG. 3, described above.

Step 408 comprises obtaining results of a biomarker determination for the patient. This may comprise analyzing an EHR for the patient to determine results of a prior biomarker measurement or calculation, or actively performing (or having performed) a biomarker measurement upon the patient (and potentially performing calculations based on the biomarker measurement), and then updating the EHR of the patient. A biomarker determination may comprise measuring a bodily fluid of the patient, such as via step 410 of drawing (or having drawn) blood and measuring (or having measured) at least one biomarker selected from the group consisting of creatinine, urea, or cystatin C. A biomarker determination may further comprise calculating estimated Glomerular Filtration Rate (eGFR) based on measurements of other biomarkers (e.g., measurements of cystatin C at laboratory 190). After the EHR of the patient has been updated with the results, it may be read by genomics server 220 or provider client 210 in order to obtain the results. In a further embodiment, results older than a threshold age (e.g., one year, six months, three months, etc.) are not considered during this process, or are ignored if more recent results are available.

Each biomarker may have a separate threshold level, and each threshold level may comprise a threshold above the average range for the general population, or a demographic subset of the population that the patient belongs to. In one embodiment, a threshold level comprises a number of standard deviations (e.g., one, two, or three standard deviations) above a mean value for the biomarker.

In one embodiment, the threshold for creatinine is 1.2 milligrams per deciliter (mg/dL), the threshold for urea is 20 mg/dL, the threshold for cystatin C is 7.3 mg/dL, and the threshold for eGFR is 90 milliliters per minute per 1.73 square meters (mL/min/1.73 m²) and greater than 60 mL/min/1.73 m². These thresholds may be stored in memory in genomics server 220 or provider client 210. In a further embodiment, values above the corresponding thresholds are indicated by corresponding medical vocabulary codes, such as ICD-10 or CPT codes. In such an instance, genomics server 220 may search for corresponding codes within an EHR of the patient.

In step 412, genomics server 220 determines whether any biomarker measurement (e.g., as identified within an EHR for the patient) is greater than the corresponding threshold, by numerical comparison or identification of relevant medical vocabulary codes. That is, the genomics server 220 determines that the biomarker meets some predetermined criteria in step 412. In an event that the biomarker level meets this predetermined criteria, processing continues to step 414, wherein the genomics server 220 determines whether the patient is a carrier of a qualifying variant in PKD1 or PKD2. If the patient is a carrier, then processing continues to step 416 wherein a PKD intervention is performed. Alternatively, if the patient is not a carrier, then processing continues to step 418, wherein genomics server 220 refrains from triggering a PKD intervention.

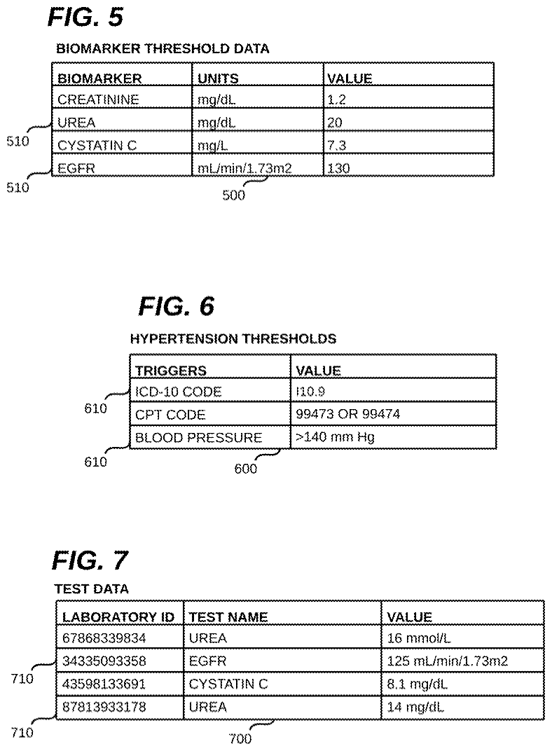

FIGS. 5-9 depict a variety of tables relating to the detection and treatment of PKD in illustrative embodiments. These tables may be stored in memory 214 of provider client 210, memory 224 of genomics server 220, or in other locations that are accessible to provider client 210 and/or genomics server 220. FIG. 5 is a table 500 that defines threshold levels for each of multiple biomarkers related to PKD. Each entry 510 defines a separate threshold for a separate biomarker. Thus, table 500 may be utilized to provide criteria for review of an EHR for values that are above these thresholds.

FIG. 6 is a table 600 for identifying the presence of hypertension in EHR data in an illustrative embodiment. Each entry 610 indicates a different technique for identifying hypertension, such as via a medical vocabulary code, or review of a direct numerical measurement of blood pressure in the EHR. Table 600 therefore provides a series of alternate criteria for reviewing an EHR to detect the condition of hypertension.

FIG. 7 is a table 700 depicting test data pertaining to PKD for each of multiple patients in an illustrative embodiment. Each entry 710 in table 700 indicates an anonymized laboratory ID for a patient, a corresponding test name, and a corresponding value. Table 700 may be created, for example, based on EHR data retrieved for patients. Laboratory IDs may be associated with EHR identifiers at genomics server 220 or provider client 210, in order to enable access to both health data and genomics data for a patient.

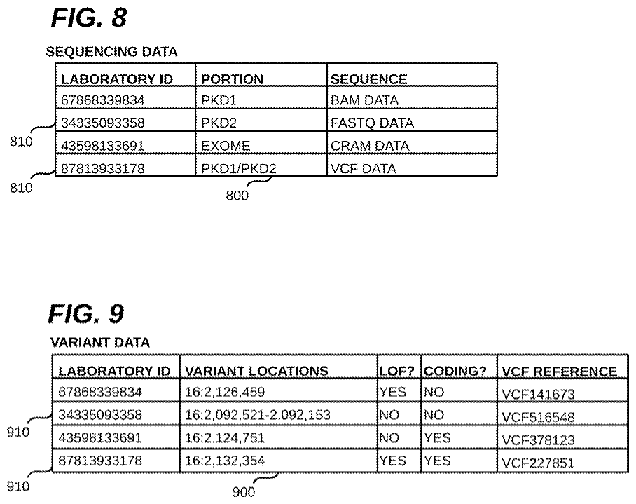

FIG. 8 is a table 800 that summarizes sequence data for individuals in an illustrative embodiment. For example, table 800 may be one of many data structures stored in genomics server 220. In this embodiment, table 800 includes an entry 810 for each of multiple patients. Each entry 810 includes a unique identifier (e.g., LSID) for the corresponding patient, as well as an indication of the gene that the sequence data relates to. The portion of the genome that has been sequenced may comprise whole genome data, whole exome data, array data, data for a specific gene or portion of a gene, etc. In this embodiment, the sequence data relates to the genes PKD1 and PKD2.

Table 800 also includes a link to sequence data for each patient, and indicates a format of the sequence data. In one embodiment, the sequence data indicates base pairs of the corresponding individual, together with the positions of those base pairs along a portion of a chromosome. In a further embodiment, the sequence data is stored as VCF and/or BED data, although any suitable technique for storing the sequence data may be utilized. In this manner, a controller 232 of the genomics server 220 may use the table 800 to rapidly identify the qualifying variants of PKD1 and/or PKD2 carried by each patient. In further embodiments, controller 232 updates table 800 to list qualifying variants of these genes detected for each patient.

FIG. 9 is a table 900 that summarizes PKD1 and PKD2 variant data for individuals in an illustrative embodiment. In this embodiment, each entry 910 in table 900 reports a location (e.g., genomic coordinate) for each genetic variant of PKD1 and/or PKD2, together with flags indicating whether the variant is an LoF or coding variant. Table 900 further includes a VCF reference, which refers to the location and/or identifier of a VCF file that indicates the presence of the variant. Table 900 may be utilized by controller 232 of genomics server 220, in order to rapidly select and report diagnostic and treatment thresholds for a patient.

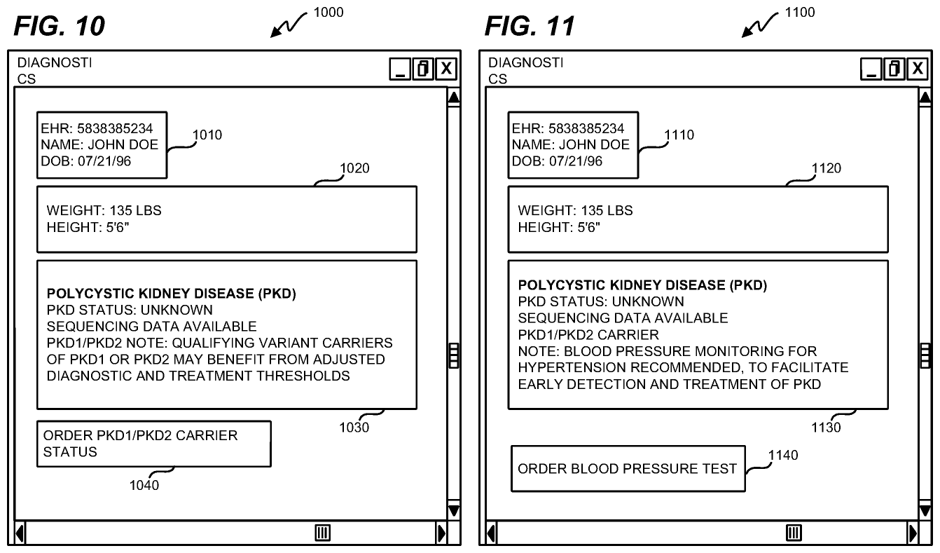

FIG. 10 depicts a Graphical User Interface (GUI) 1000 that dynamically recommends sequencing for patients that have an unknown status as a carrier of a qualifying variant of the genes PKD1 and PKD2 in an illustrative embodiment. In this embodiment, GUI 1000 includes region 1010 which provides identifying information for a patient, and region 1020 which depicts phenotypic information for the patient. Regions 1010 and 1020 may be populated, for example, by accessing data within an EHR for the patient maintained at a server accessed by provider client 210 of FIG. 2. Region 1030 provides an indication of whether PKD1/PKD2 carrier status for the patient is known, such as based on information in the EHR. In one embodiment the EHR does not include PKD1/PKD2 carrier status, and the provider client 210 transmits a message to genomics server 220 to determine whether the patient has sequencing data for PKD1 and PKD2. If genomics server 220 has this sequencing data, a medical practitioner may press button 1040 to order this information from genomics server 220 for instant delivery. Alternatively, if genomics server 220 does not have this sequencing data, a press of button 1040 may trigger an order for a blood draw or saliva sample to be provided to genomics laboratory 120 for sequencing.

FIG. 11 depicts a Graphical User Interface (GUI) 1100 that dynamically recommends additional testing, and revised diagnostic and treatment thresholds, for patients who are known to carry a qualifying variant of the gene PKD1 or the gene PKD2 in an illustrative embodiment. For example, GUI 1100 may be a variation of GUI 1000 of FIG. 10. Regions 1110 and 1120 may be populated, for example, by accessing an EHR for the patient maintained at a server accessed by provider client 210 of FIG. 2. Region 1130 provides an indication of whether PKD1/PKD2 carrier status for the patient is known. In this embodiment, the PKD1/PKD2 carrier status for the patient is both known and positive. Thus, GUI 1100 presents recommendations for implementing regular blood pressure monitoring of the patient for hypertension. Furthermore, because the PKD1/PKD2 carrier status for the patient is positive, button 1040 of FIG. 10 is replaced with button 1140 of FIG. 11. In this instance, button 1140 provides for transmission of a message that requests a blood pressure test for the patient.

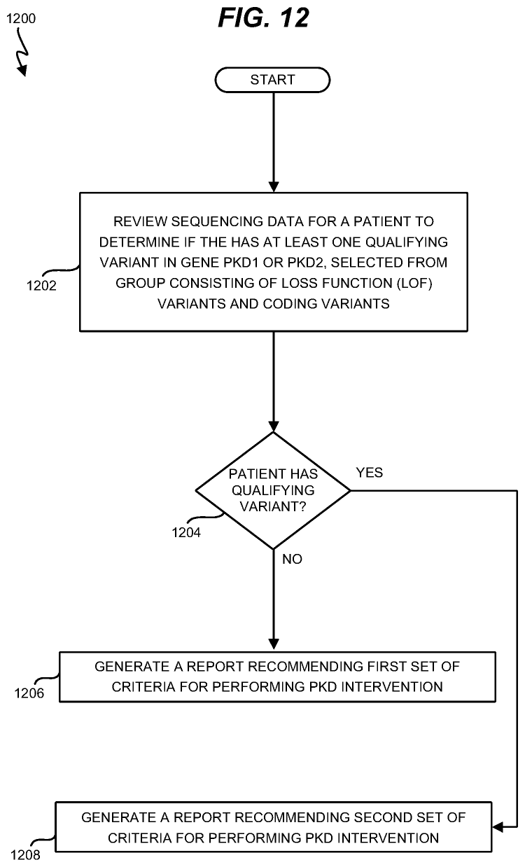

FIG. 12 is a flowchart depicting a method of reporting different criteria for PKD diagnosis and intervention, based on status as a carrier of a PKD1/PKD2 variant, in an illustrative embodiment. Method 1200 may be performed by provider client 210, genomics server 220, etc. Thus, steps 1202-1208 may be performed, for example, by controller 212 of provider client 210, or controller 232 of genomics server 220, depending on the device performing the method.

Step 1202 comprises reviewing sequencing data for the patient to determine if the patient has at least one qualifying variant in the gene PKD1 or the gene PKD2. The qualifying variant is selected from the group consisting of Loss of Function (LoF) variants and coding variants.

In step 1204, a determination is made whether the patient is a carrier of a qualifying variant of the gene PKD1 or PKD2. This may be performed according to the techniques discussed previously with regard to other methods. If the patient does not have a qualifying variant in either PKD1 or PKD2, then processing continues to step 1206, which comprises generating a report recommending a first set of criteria for performing a PKD intervention. For example, the first set of criteria may comprise imaging of the kidney confirming a polycystic condition. As used herein, a report may comprise an email, Short Message Service (SMS) notification such as a text message, an update to a GUI, or other message that presents the criteria to a user, such as a healthcare provider.

If the patient does have a qualifying variant in PKD1 or PKD2, then processing continues to step 1208, which comprises generating a report recommending a second set of criteria for performing a PKD intervention. For example, the second set of criteria may comprise a determination of hypertension for the patient.

In a further embodiment, if the patient has a qualifying variant in PKD1 or PKD2, the method further includes reviewing historic medical data for the patient. This may be performed, for example, by reviewing EHR data for the patient indicating the presence of hypertension. If the patient already has a history of hypertension, then the system may immediately trigger a PKD intervention in a similar manner to step 314 of FIG. 3.

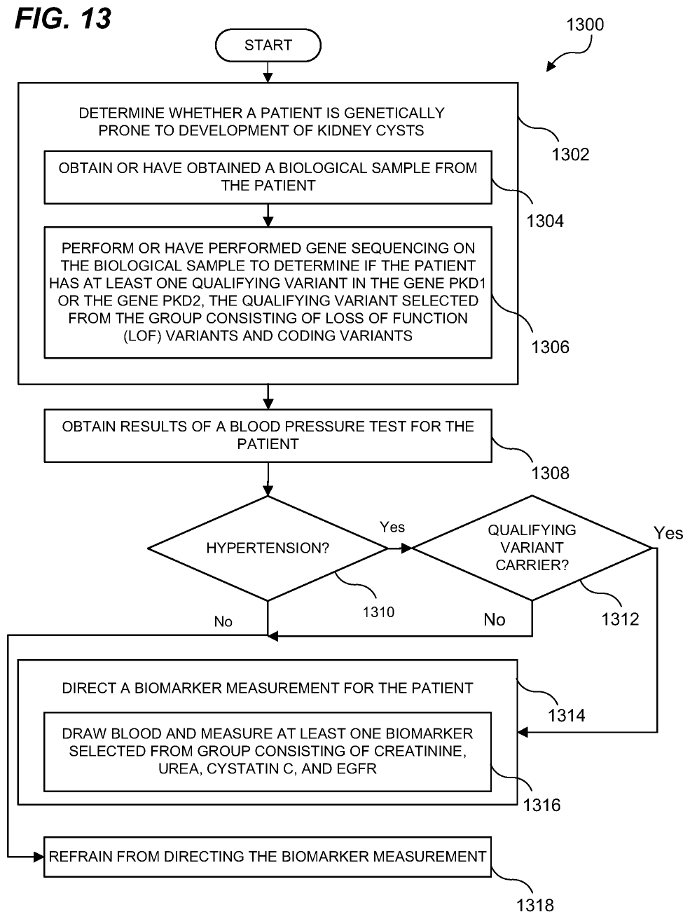

FIG. 13 provides a method 1300 of selectively performing a PKD intervention upon a patient, based on a combination of qualifying variant carrier status and a medical measurement (e.g., as measured in an EHR) in an illustrative embodiment. Specifically, method 1300 focuses on the concept of selectively performing PKD interventions based on medical data in the form of a blood pressure measurement and medical data describing biomarkers found within bodily fluid (e.g., blood samples or urine samples). Steps 1302-1306 may be performed in a similar manner to steps 302-306 of method 300 of FIG. 3, described above. In one embodiment, a determination that a patient is a carrier of a qualifying variant in PKD1 or PKD2 is made whenever a qualifying variant is called in a VCF file or similar data structure.

Step 1308 comprises obtaining results of a blood pressure test on the patient. This may comprise analyzing an EHR for the patient to determine results of a prior blood pressure test, or actively performing (or having performed) a blood pressure test upon the patient, and then updating the EHR of the patient. Again, the blood pressure test may comprise application of a pressure cuff to the patient, followed by measuring systolic and/or diastolic blood pressure for the patient via use of the cuff. In further embodiments, dedicated sensors are used to determine blood pressure. After the EHR of the patient has been updated with the results, it may be read by genomics server 220 or provider client 210 in order to obtain the results. In a further embodiment, results older than a threshold age (e.g., one year, six months, three months, etc.) are not considered during this process, or are ignored if more recent results are available.

Step 1310 comprises determining, based on the blood pressure test for the patient, whether the patient has hypertension. Again, this may comprise genomics server 220 accessing an EHR of the patient for evidence of hypertension, as indicated by a code in a medical vocabulary. In a further embodiment, step 1310 comprises actively reviewing blood pressure measurements for evidence of hypertension. In the event that the patient has grade 1 hypertension (e.g., as indicated by a systolic pressure of 130-139 millimeters of mercury (mm Hg) and/or diastolic pressure of 80-89 mm Hg), or grade 2 hypertension (e.g., as indicated by a systolic pressure of 140 mm Hg or more, and/or a diastolic pressure of greater than 90 mm Hg), processing continues to step 1312. Alternatively, if the patient does not have hypertension in step 1310, then processing continues to step 1318, wherein the genomics server 220 refrains from directing a biomarker measurement of the patient. Generally, however, any systolic pressure of above 129 mm Hg and/or any diastolic pressure of greater than 79 mm Hg may continue processing to step 1312.

In step 1312, genomics server 220 determines whether the patient is a carrier of a qualifying variant in PKD1, or a qualifying variant in PKD2 (e.g., according to the techniques and standards described above). In the event that the patient is a carrier of a qualifying variant, then processing proceeds to step 1314, to direct a biomarker measurement for the patient. Otherwise, the genomics server 220 refrains from directing a biomarker measurement of the patient, in the step 1318.

In directing a biomarker measurement for the patient, step 1314 may comprise analyzing an EHR for the patient to determine results of a prior biomarker measurement, or actively performing (or having performed) a biomarker measurement upon the patient, and then updating the EHR of the patient. A biomarker measurement may comprise measuring a bodily fluid of the patient, such as via step 1316 of drawing blood and measuring at least one biomarker selected from the group consisting of creatinine, urea, cystatin C, and/or estimated Glomerular Filtration Rate (eGFR) (e.g., at laboratory 190). After the EHR of the patient has been updated with the results, it may be read by genomics server 220 or provider client 210 in order to obtain the results. In a further embodiment, results older than a threshold age (e.g., one year, six months, three months, etc.) are not considered during this process, or are ignored if more recent results are available. Generally, the biomarker measurement of step 1316 may be performed in a manner similar to step 410 of FIG. 4.

Method 1300 provides a technical benefit over prior systems and techniques because it enables those who are genetically prone to PKD to be identified and treated prior to onset of life-altering symptoms, which in turn extends both the quantity and quality of life for such patients.

Any of the various computing and/or control elements shown in the figures or described herein may be implemented as hardware, as a processor implementing software or firmware, or some combination of these. For example, an element may be implemented as dedicated hardware. Dedicated hardware elements may be referred to as “processors,” “controllers,” or some similar terminology. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, a network processor, application specific integrated circuit (ASIC) or other circuitry, field programmable gate array (FPGA), read only memory (ROM) for storing software, random access memory (RAM), non-volatile storage, logic, or some other physical hardware component or module.

In one embodiment, instructions stored on a computer readable medium direct a computing system of any of the devices and/or servers discussed herein, such as genomics server 220, to perform the various operations disclosed herein. In some embodiments, all or portions of these operations may be implemented in a networked computing environment, such as a cloud computing system. Cloud computing often includes on-demand availability of computer system resources, such as data storage (cloud storage) and computing power, without direct active management by a user. Cloud computing relies on the sharing of resources, and generally includes on-demand self-service, broad network access, resource pooling, rapid elasticity, and measured service.

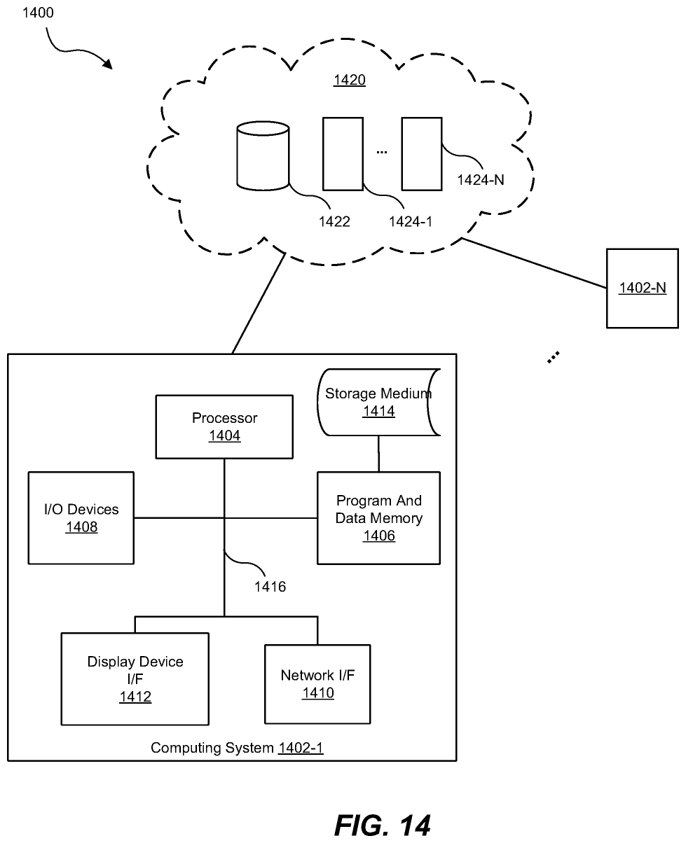

FIG. 14 depicts one illustrative cloud computing system 1400 operable to perform the above operations by executing programmed instructions tangibly embodied on one or more computer readable storage mediums. The cloud computing system 1400 generally includes the use of a network of remote servers hosted on the internet to store, manage, and process data, rather than a local server or a personal computer (e.g., in the computing systems 1402-1-1402-N). Cloud computing enables users to use infrastructure and applications via the internet, without installing and maintaining them on-premises. In this regard, the cloud computing network 1420 may include virtualized information technology (IT) infrastructure (e.g., servers 1424-1-1424-N, the data storage module 1422, operating system software, networking, and other infrastructure) that is abstracted so that the infrastructure can be pooled and/or divided irrespective of physical hardware boundaries. In some embodiments, the cloud computing network 1420 can provide users with services in the form of building blocks that can be used to create and deploy various types of applications in the cloud on a metered basis.

Various components of the cloud computing system 1400 may be operable to implement the above operations in their entirety or contribute to the operations in part. For example, a computing system 1402-1 may be used to perform analysis of gene sequencing data, and then store that analysis along with the gene sequencing data in a data storage module 1422 (e.g., a database) of a cloud computing network 1420. Various computer servers 1424-1-1424-N of the cloud computing network 1420 may be used to operate on the gene sequencing data and/or transfer the gene sequencing analysis and/or the gene sequencing data to another computing system 1402-N.

Some embodiments disclosed herein may utilize instructions (e.g., code/software) accessible via a computer-readable storage medium for use by various components in the cloud computing system 1400 to implement all or parts of the various operations disclosed hereinabove. Examples of such components include the computing systems 1402-1-1402-N.

Exemplary components of the computing systems 1402-1-1402-N may include at least one processor 1404, a computer readable storage medium 1414, program and data memory 1406, input/output (I/O) devices 1408, a display device interface 1412, and a network interface 1410. For the purposes of this description, the computer readable storage medium 1414 comprises any physical media that is capable of storing a program for use by the computing system 1402. For example, the computer-readable storage medium 1414 may be an electronic, magnetic, optical, electromagnetic, infrared, semiconductor device, or other non-transitory medium. Examples of the computer-readable storage medium 1414 include a solid-state memory, a magnetic tape, a removable computer diskette, a random access memory (RAM), a read-only memory (ROM), a rigid magnetic disk, and an optical disk. Some examples of optical disks include Compact Disk-Read Only Memory (CD-ROM), Compact Disk-Read/Write (CD-R/W), Digital Versatile Disc (DVD), and Blu-Ray Disc.

The processor 1404 is coupled to the program and data memory 1406 through a system bus 1416. The program and data memory 1406 include local memory employed during actual execution of the program code, bulk storage, and/or cache memories that provide temporary storage of at least some program code and/or data in order to reduce the number of times the code and/or data are retrieved from bulk storage (e.g., a hard disk drive, a solid state drive, or the like) during execution.

Input/output or I/O devices 1408 (including but not limited to keyboards, displays, touchscreens, microphones, pointing devices, etc.) may be coupled either directly or through intervening I/O controllers. Network adapter interfaces 1410 may also be integrated with the system to enable the computing system 1402 to become coupled to other computing systems or storage devices through intervening private or public networks. The network adapter interfaces 1410 may be implemented as modems, cable modems, Small Computer System Interface (SCSI) devices, Fibre Channel devices, Ethernet cards, wireless adapters, etc. Display device interface 1412 may be integrated with the system to interface to one or more display devices, such as screens for presentation of data generated by the processor 1404.

What is claimed is:

1. A method for selectively treating a patient for Polycystic Kidney Disease (PKD), the method comprising:
determining whether the patient is genetically prone to development of kidney cysts, by:
obtaining or having obtained a biological sample from the patient; and
performing or having performed sequencing on the biological sample to determine if the patient has at least one qualifying variant in either a gene PKD1 or a gene PKD2, the qualifying variant selected from the group consisting of Loss of Function (LoF) variants and coding variants; and
obtaining results of a blood pressure test upon the patient;
if the results of the blood pressure test indicate hypertension for the patient:
if the patient has a qualifying variant in the gene PKD1 or the gene PKD2, triggering a PKD intervention; and
if the patient does not have a qualifying variant in the gene PKD1 or the gene PKD2, refraining from triggering the PKD intervention.

2. The method of claim 1 further comprising:
reviewing an Electronic Health Record (EHR) of the patient to determine whether the results of the blood pressure test indicate hypertension for the patient.

3. The method of claim 2 wherein:
reviewing the EHR comprises searching the EHR for at least one item selected from the group consisting of: a medical vocabulary code indicating hypertension, a systolic blood pressure measurement above 129 millimeters of mercury, and a diastolic blood pressure measurement above 79 millimeters of mercury.

4. The method of claim 1 wherein the PKD intervention is selected from the group consisting of:
prescribing angiotensin-converting enzyme (ACE) inhibitors, prescribing angiotensin II receptor blockers, prescribing diuretics, prescribing erythropoietin, prescribing statins, prescribing calcium, prescribing vitamin D supplements, prescribing tolvaptan, and implementing a protein-reduced diet.

5. The method of claim 1 wherein:
the blood pressure test is performed before the sequencing, and results of the blood pressure test are stored in an Electronic Health Record (EHR) for the patient.

6. The method of claim 1 further comprising:
in response to the sequencing, generating a report indicating that the PKD intervention be performed for the patient if the patient is later determined to have hypertension.

7. The method of claim 1 further comprising:
triggering the PKD intervention by generating a report recommending the PKD intervention.

8. A method for selectively treating a patient for Polycystic Kidney Disease (PKD), the method comprising:
determining whether the patient is genetically prone to development of kidney cysts, by:
obtaining or having obtained a biological sample from the patient; and
performing or having performed sequencing on the biological sample to determine if the patient has at least one qualifying variant in either a gene PKD1 or a gene PKD2, the qualifying variant selected from the group consisting of Loss of Function (LoF) variants and coding variants; and
obtaining results of a biomarker determination for the patient;
if the results of the biomarker determination meet a predefined criteria:
if the patient has a qualifying variant in the gene PKD1 or the gene PKD2, triggering a PKD intervention; and
if the patient does not have a qualifying variant in the gene PKD1 or the gene PKD2, refraining from triggering the PKD intervention.

9. The method of claim 8 further comprising:
reviewing an Electronic Health Record (EHR) of the patient to determine the results of the biomarker measurement.

10. The method of claim 8 wherein the PKD intervention is selected from the group consisting of:
prescribing angiotensin-converting enzyme (ACE) inhibitors, prescribing angiotensin II receptor blockers, prescribing diuretics, prescribing erythropoietin, prescribing statins, prescribing calcium, prescribing vitamin D supplements, prescribing tolvaptan, and implementing a protein-reduced diet.

11. The method of claim 8 wherein:
the biomarker determination is performed before the sequencing, and results of the biomarker determination are stored in an Electronic Health Record (EHR) for the patient.

12. The method of claim 8 further comprising:
in response to the sequencing, generating a report indicating that the PKD intervention be performed for the patient if the patient is later determined to have a biomarker amount higher than the predefined threshold.

13. The method of claim 8 wherein:
the biomarker is selected from the group consisting of creatinine, urea, cystatin C, and estimated Glomerular Filtration Rate (eGFR).

14. The method of claim 13 wherein:
the biomarker is eGFR, and the predefined criteria are defined as a calculation that indicates less than 90 milliliters per minute per 1.73 square meters, and greater than 60 milliliters per minute per 1.73 square meters.

15. The method of claim 13 wherein:
the biomarker is urea, and the predefined criteria are defined as exceeding 20 milligrams per deciliter.

16. The method of claim 13 wherein:
the biomarker is cystatin C, and the predefined criteria are defined as exceeding 7.3 milligrams per deciliter.

17. The method of claim 13 wherein:
the biomarker is creatinine, and the predefined criteria are defined as exceeding 1.2 milligrams per deciliter.

18. A method for selectively recommending treatment for a patient for Polycystic Kidney Disease (PKD), the method comprising:
reviewing sequencing data for the patient to determine if the patient has at least one qualifying variant in a gene PKD1 or a gene PKD2, the qualifying variant selected from the group consisting of Loss of Function (LoF) variants and coding variants;
if the patient does not have a qualifying variant in the gene PKD1 or the gene PKD2:
generating a report recommending a first set of criteria for performing a PKD intervention; and
if the patient does have a qualifying variant in the gene PKD1 or the gene PKD2:
generating a report recommending a second set of criteria for performing the PKD intervention.

19. The method of claim 18 wherein:
the first set of criteria comprises confirmation of a polycystic condition; and
the second set of criteria comprises a determination of hypertension for the patient.

20. The method of claim 18 wherein the PKD intervention is selected from the group consisting of:
prescribing angiotensin-converting enzyme (ACE) inhibitors, prescribing angiotensin II receptor blockers, prescribing diuretics, prescribing erythropoietin, prescribing statins, prescribing calcium, prescribing vitamin D supplements, prescribing tolvaptan, and implementing a protein-reduced diet.

21. A method for selectively treating a patient for Polycystic Kidney Disease (PKD), the method comprising:
determining whether the patient is genetically prone to development of kidney cysts, by:
obtaining or having obtained a biological sample from the patient; and
performing or having performed sequencing on the biological sample to determine if the patient has at least one qualifying variant in either a gene PKD1 or a gene PKD2, the qualifying variant selected from the group consisting of Loss of Function (LoF) variants and coding variants;
obtaining results of a blood pressure test upon the patient; and
if the results of the blood pressure test indicate hypertension for the patient:
if the patient has a qualifying variant in the gene PKD1 or the gene PKD2, directing a biomarker determination for the patient, the biomarker determination selected from a group consisting of a creatinine measurement, a urea measurement, a cystatin C measurement, and an estimated Glomerular Filtration Rate (eGFR) calculation for blood measurements from the patient; and
if the patient does not have a qualifying variant in the gene PKD1 or the gene PKD2, refraining from directing the biomarker measurement.
